(19) United States
(12) Patent Application Publication
(10) Pub. No.: US 2005/0138680 A1
(43) Pub. Date: Jun. 23, 2005

(54) Method for generating non-human mammalian chimeric embryo

(75) Inventors: Kun-Hsiung Lee, Miaoli County (TW); Hut-Wen Wang, Miaoli County (TW); Hui-Rong Chang, Miaoli County (TW); Ching-Fu Tu, Miaoli County (TW); Chih-Jen Lin, Miaoli County (TW)

Correspondence Address: BACON & THOMAS, PLLC, 625 SLATERS LANE, FOURTH FLOOR, ALEXANDRIA, VA, 22314, US

(73) Assignee: ANIMAL TECHNOLOGY INSTITUTE TAIWAN, Miaoli (TW)

(21) Appl. No.: 10/875,225

(22) Filed: Jun. 25, 2004

(30) Foreign Application Priority Data
Dec. 22, 2003 (TW) 92136457

(51) Int. Cl. A01K 67/027

(52) U.S. Cl. 800/21

(57) ABSTRACT

The present invention relates to a method for generating non-human mammalian chimeric embryo. The method involves coculturing denuded non-human mammalian embryos with cells in an Eppendorf micro test tube. The chimeric embryo obtained is then transferred into a non-human recipient mammal so as to develop into a non-human chimeric fetus, non-human chimeric mammal, an embryonic stem cell-derived fetus or an embryonic stem cell-derived mammal.

### FIELD OF THE INVENTION

The present invention relates to a method for generating non-human mammalian chimeric embryos. Specifically, it relates to coculture denuded (i.e. zona pellucida-free) embryos with embryonic stem, ES, cells in an Eppendorf micro test tube so as to obtain non-human mammalian chimeric embryos. The present invention also relates to a method for generating non-human chimeric fetus and mammals, wherein the foregoing derived chimeric embryos were further in vivo grown into non-human chimeric fetus and mammals.

### DESCRIPTION OF THE RELATED ARTS

The transgenic animal could be generated by microinjecting an exogenous gene into a pronucleus embryo, and allowing the gene to be incoporated randomly into the genome of the embryo itself. The establishment of a transgenic animal allows accurate and efficient investigation of the in vivo temporal and spatial functions of almost all genes from the embryonic, fetal, and perinatal stages until adulthood. Transgenic animal model was first successfully established in mouse since establishing a transgenic mouse requires less time and effort than that of other large-sized mammals. Transgenic mouse therefore becomes an intensively used animal models in the field of life science.

DNA pronucleus microinjection is one option for creating a transgenic animal. The problem of this method is that the exogenous DNA sequence randomly inserts into multiple sites or single site in the chromosomal DNA. Therefore it is very often that multiple lines of transgenic mice are needed to establish for one DNA sequence pronucleus microinjection to ascertain the results. This method is relatively easy to carry out, but the follow-up breeding, maintenance, and research are rather laborious.

The embryonic stem (ES) cell system has been employed for gene targeting and a subsequent production of a transgenic animal. By establishing just one genetic targeted, germline transmitted male transgenic animal, its progenies may as well carry the transgene, thus making the follow-up studies pretty time and labor-saving. Transgenic animals established through the ES cell system have been proved to be very efficient for investigating the in vivo temporal and spatial physiological functions and mechanisms of almost all DNA sequences. Despite the steps of homologous recombination and subclone confirmation are time-consuming, the techniques are well developed and thus a chimeric animal is not difficult to be generated. The current bottlenecks are the inconsistent ratio and a lengthy time period for comfirmation of germline transmission of a transgenic animal, posing a thorny problem to many reasearchers. It is obvious that the generation of chimeric animal capable of efficient germline transmission is a crucial key to the problem.

The generation of germline competent chimeric mice via embryonic stem (ES) cells and ES cell-derived mice is a crucial step in developing gene-manipulated mouse models. To date, techniques for generating chimeric mice include the direct microinjection of ES cells into the blastocoel of 3.5 days post coitum (dpc) blastocysts and aggregation (Bradley, 1987; Wood et al., 1993a; Hogan et al., 1994; Nagy et al., 2003; Lee, 1992; Lee et al., 2003) as well as coculture 2.5 dpc denuded embryos with ES cells (Wood et al., 1993b; Suzuki et al., 1994; Ueda et al., 1995; Shimada et al., 1999). Although these methods are good enough to generate chimeric embryos, they have various advantages and disadvantages.

Microinjection mainly involves injecting ES cells directly into the blastocoel of 3.5 dpc blastocysts. This is a very efficient and highly repeatable method. However, this method suffers various limitations. First, the micromanipulation equipments are expensive. Second, intensive training is required to master the required micromanipulation skills. Third, the microinjection itself is time-consuming, averaging approximately 20-40 blastocysts per hour (Bradley, 1987; Hogan et al., 1994; Nagy et al., 2003). Due to the above mentioned-limitations, microinjection is often entrusted to a specialized organization to carry out. Notwithstanding its efficiency in generating chimeric mouse, the inconsistent rate in obtaining germline transmitted chimeric mouse and the follow-up laborious screening and confirmation procedures pose the major obstacles to mass production.

Previous studies concerning an alternative that involves microinjecting ES cells into 2.5 dpc 8-cell embryos have been reported with various results. Papaioannou and Johnson (1993, 2000) have reported that the result was the comparable to blastocoel of 3.5 dpc blastocysts, while Tokunaga and Tsunoda (1992) have demonstrated that the ratio of male chimeric mouse and which capable of germline transmission increased significantly. It is to be noted that manipulating microinjection with 8-cell embryo is much more difficult than 3.5 dpc blastocyst and is thus rarely employed.

Aggregation requires no expensive and sophisticated instruments, and is easy to learn and implement. It is based on the sticky characteristics of the zona pellucida-free (denuded) embryo and ES cell, those allow them to adhere to each other and develop into a chimeric embryo. Aggregation is mainly performed with single 2.5 dpc denuded embryo (Khillan and Bao, 1997; Kondoh et al., 1999) or a set of double 2.5 dpc denuded embryos (Bradley, 1987, Wood et al., 1993a; Shimada et al., 1999) between the 8-cell and morula stage. The major problem in the former method (single embryo) is its low efficiency (Khillan and Bao, 1997; Kondoh et al., 1999), while in the later (double embryos) is that two embryos (either XX or XY) are required to create a single chimeric embryo, which is obviously unfavorable for inbred mice since only about 4-5 embryos are recovered per mouse through natural mating.

Still another method for generating chimeric embryos is coculture. It is simply performed by coculturing 2.5 dpc denuded 8-cell to morula stage embryos with ES cell on culture dish surface (Woods et al., 1993b; Shimada et al., 1999) or in the same droplet (Ueda et al., 1995). However, this method is less efficient as compared with the abovementioned methods (Suzuki et al., 1994; Ueda et al., 1995).

U.S. Pat. No. 5,449,620 and No. 6,281,408 disclose two methods and apparatus for generating chimeric embryos and chimeric mice as well. The former teaches a method and apparatus for aggretation in a tapering depression; as for the later, it provides a method of producing compound transgenic animals by coculturing embryonic stem cells with a denuded morula in a microwell plate. The techniques in the foregoing two patents require being handled set by set, which is unfaborable for mass production.

In conclusion, despite various methods for generating chimeric embryos and chimeric animals are available to date, bottlenecks such as expensive instruments, intensive training, sophisticated techniques, or variable results have posed major obstables for mass production. Therefore, there still exists a long-felt need in the art for a relative simple, reliable, mass producible, and effective method that can not only overcome the above obstacles but also reaches higher efficiency for generating chimeric embryos and chimeric animals.

### SUMMARY OF THE INVENTION

In light of the foregoing drawbacks in the prior art, one object of the present invention is to provide a simple and effective method for generating a non-human mammalian chimeric embryo. In one aspect, the method of the present invention comprises the following steps: providing a cell; providing a denuded (zona pellucida-free) non-human mammalian embryo that is from 1-cell stage to morula stage; mixing and coculturing the foregoing cell and denuded non-human mammalian embryo in physiological medium in an Eppendorf micro test tube to obtain a non-human mammalian embryo-cell aggregate; and continuing culturing said non-human mammalian embryo-cell aggregate in physiological medium so as to generate a non-human mammalian chimeric embryo.

Another object of the present invention is to provide a method for generating non-human chimeric mammal, comprising the steps of: providing a cell; providing a denuded non-human mammalian embryo that is from 1-cell stage to morula stage; mixing and coculturing the foregoing cell and denuded non-human mammalian embryo in physiological medium in an Eppendorf micro test tube to obtain a non-human mammalian embryo-cell aggregate; continuing culturing the foregoing non-human mammalian embryo-cell aggregate in physiological medium so as to generate a non-human mammalian chimeric embryo; transferring the foregoing non-human mammalian chimeric embryo into a non-human recipient mammal; and allowing the transferred chimeric embryo to grow in the foregoing non-human recipient animal so as to develop to term as a non-human chimeric mammal or an embryonic stem cell-derived mammal.

Still another object of the present invention is to provide a method for generating non-human chimeric fetus, comprising the steps of: providing a cell; providing a denuded non-human mammalian embryo that is from 1-cell stage to morula stage; mixing and coculturing the foregoing cell and denuded non-human mammalian embryo in physiological medium in an Eppendorf micro test tube to obtain a non-human mammalian embryo-cell aggregate; continuing culturing the foregoing non-human mammalian embryo-cell aggregate in physiological medium so as to generate a non-human mammalian chimeric embryo; transferring the foregoing non-human mammalian chimeric embryo into a non-human recipient mammal; and allowing the transferred chimeric embryo to grow in the foregoing non-human recipient animal so as to develop into a non-human chimeric fetus or an embryonic stem cell-derived fetus.

The method of the present invention allows the non-human mammalian chimeric embryo to be successfully implanted into a non-human recipient mammal. Therefore a non-human chimeric fetus, mammal or an embryonic stem cell-derived fetus, mammal can be developed from the foregoing non-human mammalian chimeric embryo generated with the foregoing Eppendorf micro test tube coculture method.

### BRIEF DESCRIPTION OF THE DRAWINGS

These and other features, aspects and advantages of the present invention will become better understood with regard to the following description, appended claims and accompanying drawings that are provided only for further elaboration without limiting or restricting the present invention, where:

FIG. 1A shows mouse ES cell, ESC 26GJ9012-8-2 that expresses green fluorescent protein, being trypsinized by 0.25% trypsin into single cell suspension.

FIG. 1B represents the same view with FIG. 1A under fluorescence microscopy.

FIG. 2A shows that the mouse ES cells, ESC 26GJ9012-8-2, expresses green fluorescent protein after being purified by standing in a CO₂ incubator for 80 min.

FIG. 2B represents the same view with FIG. 2A under fluorescence microscopy.

FIG. 3A shows that the freshly thawed mouse ES cells, ESC 26GJ9012-8-2, expresses green fluorescent protein after being purified by successive standing twice in a CO₂ incubator.

FIG. 3B represents the same view with FIG. 3A under fluorescence microscopy.

FIG. 4A shows the embryo-cell aggregates recovered from the bottom of an Eppendorf micro test tube after 2-hour coculture of the denuded embryos and the purified mouse ES cells, ESC 26GJ9012-8-2.

FIG. 4B represents the same view with FIG. 4A under fluorescence microscopy.

FIG. 5A shows the chimeric embryos developed from the embryo-cell aggregates in FIG. 4A after overnight incubation.

FIG. 5B represents the same view with FIG. 5A under fluorescence microscopy.

FIG. 6A shows the chimeric blastocysts developed from the chimeric morulas as shown in FIG. 5A after another overnight incubation.

FIG. 6B represents the same view with FIG. 6A under fluorescence microscopy.

FIG. 7A shows 3 chimeric mice naturally born after the chimeric blastocytsts being transferred into the oviduct of the ICR pseudopregnant female mouse.

FIG. 7B represents the same mice with FIG. 7A under fluorescence microscopy.

FIG. 8A shows that 9 in the 14 mice express green fluorescence under fluorescence microscopy. These mice are offsprings of a green fluorescent chimeric male mouse backrossing with a ICR female mouse.

FIG. 8B shows the 9 green fluorescent mice of FIG. 8A under fluorescence microscopy.

FIG. 9A shows the embryo-cell aggregates recovered from the bottom of an Eppendorf micro test tube after 2-hour coculture of 4n denuded embryos with purified mouse ES cells, ESC 26GJ9012-8-2.

FIG. 9B represents the same view with FIG. 9A under fluorescence microscopy.

FIG. 10A shows the 4n chimeric embryos developed from 4n embryo-cell aggregates in FIG. 9A after overnight incubation.

FIG. 10B represents the same view with FIG. 10A under fluorescence microscopy.

FIG. 11A shows the 4n chimeric blastocysts developed from the chimeric morulas as shown in FIG. 10A after another overnight incubation.

FIG. 11B represents the same view with FIG. 11A under fluorescence microscopy.

### DETAILED DESCRIPTION OF THE INVENTION

The method of the present invention provides simple and effective method for generating non-human mammalian chimeric embryo, fetus, and mammal, which can be achieved simply by coculturing cells and denuded embryos in an Eppendorf micro test tube. In one embodiment, the mouse embryonic stem (ES) cells and that express green fluorescence are used for convenience of observation. The foregoing green fluorescent ES cells were established previously (Lee et al., 2003). The green fluoresce enables in vivo expression and permits a long-term observation and monitor.

Specifically, the present method for generating a non-human mammalian chimeric embryo comprises the following steps: providing a cell; providing a denuded non-human mammalian embryo that is from 1-cell stage to morula stage; mixing and coculturing the foregoing cell and denuded non-human mammalian embryo in physiological medium in an Eppendorf micro test tube to obtain a non-human mammalian embryo-cell aggregate; and continuing culturing said non-human mammalian embryo-cell aggregate in physiological medium so as to generate a non-human mammalian chimeric embryo.

It is also specific that the foregoing method may further comprise the following steps: transferring the foregoing non-human mammalian chimeric embryo into a non-human recipient mammal; and allowing the aforesaid transferred chimeric embryo to grow in said non-human recipient mammal so as to develop into an offspring.

It is more specific that the above-mentioned offspring can be a non-human chimeric fetus, an embryonic stem cell-derived fetus, a non-human chimeric mammal or an embryonic stem cell-derived mammal.

More specifically, in the coculture step in Eppendorf micro test tube, suitable physiological medium can be chosen according the experimental conditions. Examples of the suitable physiological medium includes, but not limited to: STO, KSOM, KSOM-AA, CZB, M16 medium and combination thereof, wherein the foregoing physiological medium may be further supplemented with serum or binder, such as lectins.

It is even more specific that in the step of culturing the foregoing non-human embryo-cell aggregate or non-human mammalian chimeric embryo, suitable physiological medium can be chosen according the experimental conditions. Examples of the suitable physiological medium used for includes, but not limited to: STO, KSOM, KSOM-AA, CZB, M16 medium and combination thereof, wherein the foregoing physiological medium may be further supplemented with serum.

Particularly, the foregoing cell used in the method of the present invention may be treated or untreated with purification procedures. The genetic material of the cell may either undergo modification or not, such as random insertion or gene targeting. Furthermore, the cell may be either a primary cell or obtained from a cell line.

More particularly, the foregoing denuded non-human mammalian embryo may be obtained via in vivo growth, in vitro culture system, or a combination thereof. Furthermore, the cell and denuded non-human mammalian embryo can be of the same species or different species animals.

It is even more particular that the foregoing chimeric embryo may be transferred into the oviducts, uterus, or uterine horn of a non-human recipient mammal.

It is more specific that the foregoing Eppendorf micro test tube can be a sterilizable container which may be treated with sterilization processes, for example, but not limited to autoclave, γ-ray radiation, alcohol sterilization, UV exposure, or dry-heat sterilization. The Eppendorf micro test tube may be any container with no limits in sizes and specifications; however, it is more preferable that a commercially available 1.5 mL sterilizable Eppendorf micro test tube is applied in the present invention.

Definitions

It must be noted that as used herein and in the appended claims, the singular forms “a”, “an”, and “the” include plural reference unless the context clearly dictates otherwise. Thus, for example, reference to “a chimeric embryo” includes a plurality of such chimeric embryos, reference to “the ES cell” is a reference to one or more ES cells and equivalents thereof known to those skilled in the art, and so forth.

The term “denuded embryo” used herein refers to the embryo with its zona pellucida being removed. In the method of the present invention, the denuded embryo may ranges from 1-cell stage to morula stage, i.e., any of a 1-cell to 8-cell stage embryo or a morula stage embryo may be utilized according to the present invention unless the context clearly dictates otherwise. The foregoing denuded embryo may comprise diploid (2n) or multiploid chromosome, such as, but not limited to tetraploid (4n).

The term “mammal” used herein refers to the higher vertebrate as defined in Webster's Medical Desk Dictionary 407 (1986), and includes all members of the Mammalia class. The method of the present invention is applicable to any of these mammals except human.

The term “fetus” used herein refers to an unborn or unhatched vertebrate, particularly of a mammal, after attaining the basic structural plan of its kind.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### EXAMPLE 1

### Generating Chimeric Mouse Embryo using the Method of the Present Invention

Source of the Mice, Housing, and Feeding Evnironment

The mice were obtained from the Laboratory Animal Center, National Taiwan University College of Medicine and the National Laboratory Animal Center. The housing, feeding, superovulation, mating, and surgery were carried out according “A Guidebook for the Care and Use of Laboratory Animals” published by the Chinese Association of Laboratory Animal Science in 2001. The use of mice has been approved by this Institutional Animal Care and Use Committee.

The mice was raised in a clean conventional rodent animal facility. The air, pressure, lighting, and temperature of each animal room were independently controlled. The positive pressure and fresh air of each room were maintained by HEPA filters and pressure releaser. The light and dark cycle (14L:10D) in the animal room were controlled automatically. Temperature (18-26° C.) and relative humidity control were controlled with thermostatic devices including separate air-conditioner and heater with autosensor device. Mice were kept in autoclaved standard mouse cages supplied with bedding material and fed with high quality feeding stuff and bottled, autoclaved water. These materials were changed once or twice per week.

Coculture of Embryos and Cells

The conditions for establishing mouse ES cell line of the present invention is as described by Lee (1992) and Lee et al. (2003), with little modification in the culture medium as shown in Table 1. Mouse embryos were cultured in modified KSOM-AA medium (Table 2) in a 5% CO₂ incubator (Erbach et al., 1994; Biggers et al., 2000), while when cultured outside the CO₂ incubator, KSOM-AA medium was futher supplemented with 20.85 mM HEPES (Sigma H 6147). The composition of STO medium (4,500 mg glucose/L DMEM+10% FBS+1% penicillin-streptomycin)and STO feeder cells were prepared according to Lee (1992) and Lee et al. (2003).

TABLE 1
The composition of ES cell medium for
establishing and culturing mouse ES cells^{d,e}

| | Brand and | | | |
|---|---|---|---|---|
| Ingredient | Cat. No. | Concentration | Dosage | |
| DMEM (4,500 mg | Sigma D 6780, | | 1 | pack |
| glucose/L) | D 7777 | | | |
| Non-essential | Sigma M 7145 | 1.0% | 13.2 | mL |
| amino acids | | | | |
| β | Sigma M 6250 | 0.1 mM^{a} | 13.2 | mL |
| -mercaptoethanol | | | | |
| Leukemia | Chemicon | | 10⁶ | units |
| inhibitory | LIF2010, | | | |
| factor^{b} | StemCell Tech. | | | |
| | 02740 | | | |
| penicillin- | Gibco | 1.0% | 13.2 | mL |
| streptomycin | 15070-014 | | | |
| NaHCO₃ | Gibco | 33.88 mM | 3.7 | g |
| | 11810-025 | | | |
| fetal bovine | (HyClone | 20.0%^{c} | 260.0 | mL |
| serum (FBS) | defined and | | | |
| | tested batches | | | |
| | or ES cell | | | |
| | grade) | | | |
| dd H₂O | | | 1,000.0 | mL |
| Total volume | | | 1,299.6 | mL |

^{a}For preparation of 0.1 mM β-mercaptoethanol: dissolve 10 μL β-mercaptoethanol in 14.3 mL PBS.
^{b}The leukemia inhibitory factor (LIF) can be omitted depends on the condition of STO feeder.
^{c}Could be 15% for routine maintenance.
^{d}Adjust to pH 7.1 with 5 N HCl or 5N NaOH. The osmolarity should be 320 ± 15 mOsm/kg H₂O. Filter sterilize (0.25 μm) and store at 4° C. Warm to around 30° C. before use.
^{e}1% L-glutamine (Gibco Cat. No. 25030-081; 200 mM, 29.2 mg/mL) should be supplemented every 2-3 weeks.

TABLE 2
The composition of the modified KSOM-AA medium
used for culturing mouse embryos^{a}

| | Brand and | Concentration | Dosage |
|---|---|---|---|
| Ingredient | Cat. No. | (mM) | (g/L) |
| NaCl | Sigma S 5886 | 95.00 | 5.553 |
| KCl | Sigma P 5405 | 2.50 | 0.186 |
| KH₂PO₄ | Sigma P 5655 | 0.35 | 0.048 |
| MgSO₄ .7H₂O | Sigma M 7774 | 0.20 | 0.049 |
| sodium lactate | Sigma L 7900 | 10.00 | 1.870 mL |
| | (60% syrup) | | |
| Glucose | Sigma G 6152 | 0.20 | 0.036 |
| Penicillin | Sigma P 4687 | 100 units/mL | 0.060 |
| Streptomycin | Sigma S 1277 | | 0.050 |
| sodium | Sigma P 4562 | 0.20 | 0.022 |
| pyruvate | | | |
| NaHCO₃ | Sigma S 5761 | 25.00 | 2.100 |
| CaCl₂ .2H₂O | Sigma C 7902 | 1.71 | 0.252 |
| L-glutamine | Gibco | 1.00 | 5.000 mL |
| | 25030-081 | | |
| EDTA.2Na.2H₂O | Sigma E 6635 | 0.01 | 0.004 |
| BSA (Fr. V) | Sigma A 3311 | | 1.000 |
| MEM NEAA | Sigma M 7145 | | 5 mL |
| MEM EAA | Sigma M 5550 | | 10 mL |
| dd H₂O | | | 984.13 mL |

^{a}KSOM medium can be prepared using stocks with a composition like that of M16 stocks. Adjust to pH 7.0 with 0.5 N HCl or 0.5 N NaOH. The osmolarity should be 275 ± 15 mOsm/kg H₂O. Filter sterilize (0.25 μm) and store at 4° C. for up to 10 days.

Purification of Mouse ES Cell and ESC 26GJ9012-8-2 Expressing Green Fluorescence

Mouse ES cell line, ESC 26, were derived from the 3.5 dpc blastocysts collected from superovulated, albino (cc), inbred BALB/c female mouse after natural mating with a albino (cc) or light chinchilla (cc^{ch}), inbred 129/SvJ male mouse (purchased from The Jackson Laboratory, US) (Lee et al, 2003). The ESC 26 has been transfected with pCX-EGFP (Niwa et al 1991; Okabe et al., 1997) and ESC 26GJ9012-8-2 subcloned (Lee et al., 2003).

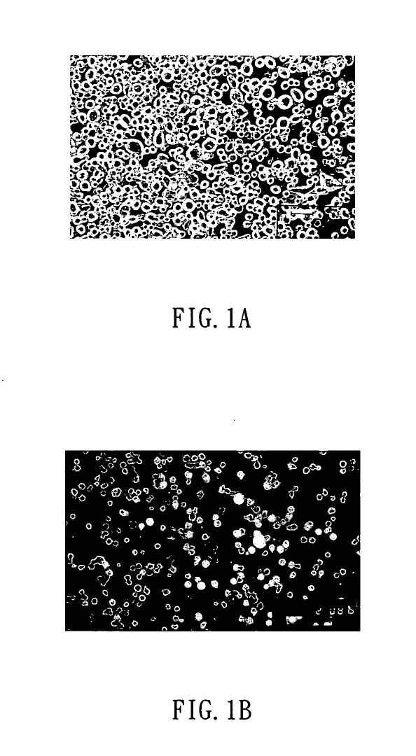

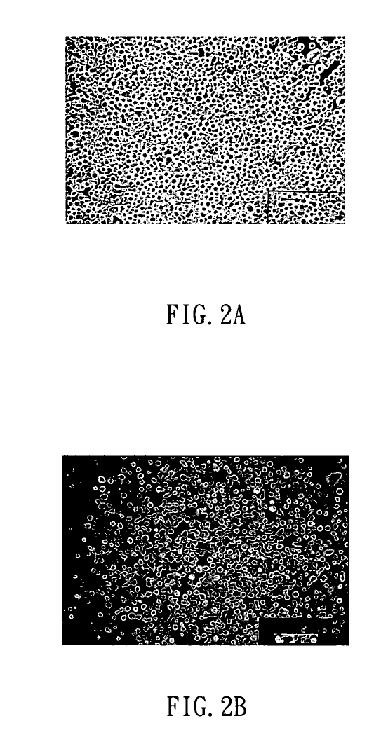

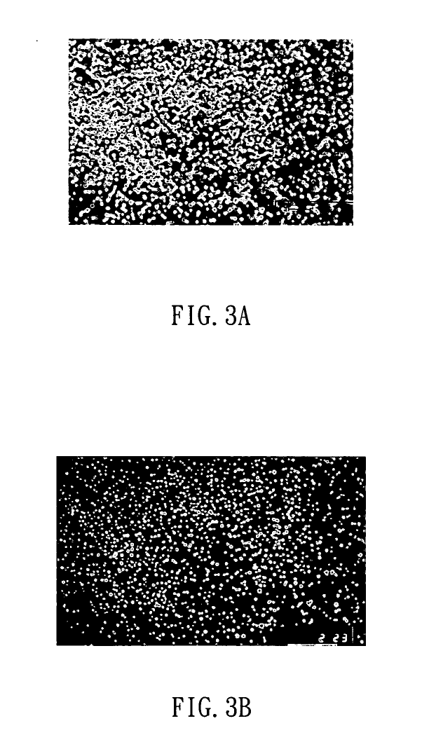

The ESC 26GJ9012-8-2 cells that seeded on a 35-mm dish with STO feeder and grown for about 1.5±0.5 days were trypsinized to a single cell suspension as shown in FIG. 1A, wherein the scale is 50 μm. FIG. 1B shows the same view with FIG. 1A under fluorescence microscopy with the scale being 100 μm. FIG. 1B shows the percentage of ES cells expressing green fluorescence was not very high. Then, about half of the cell suspension was transferred to a new, blank 60-mm culture dish containing ESC medium, and stood in 37° C., 5% CO₂ incubator for about 80 minutes. The unattached suspended cells, most of which are low in viability or dead, were sucked out, and the attached or attaching cells (˜94% were green fluorescent expressing ES cells) were washed off gently with 2 mL ESC medium. The cells were observed under a microscope, and the results are shown in FIGS. 2A and 2B, both of them show the same view with the scale being 50 μm in FIG. 2A and 100 μm in FIG. 2B. The percentage of ES cells expressing green fluorescence in FIGS. 2A and 2B was significantly increased than that in FIGS. 1A and 1B. The cells were again transferred to a new, blank 60-mm culture dish and incubated for another 20 minutes. The unattached suspended cells, more than 96% of which are green fluorescent expressing ES cells, were harvested and centrifuged twice at 173×g for 3 minutes. STO medium was added to adjust cells to a final concentration of about 4.0±1.0×10⁵/mL. The final harvested cells expressing green fluorescence can reach as high as 97% in average through this approach. Morever, freshly thawed mouse ES cell, ESC 26GJ9012-8-2, can also be purified by adopting double plating method, whereby the cell suspension was first stood for 100 minutes and followed by the second stood for 30 minutes, so as to obtain highly purified ES cells expressing green fluorescence as shown in FIG. 3A and FIG. 3B, both show the same view with the scale being 100 μm in FIG. 3A and 200 μm in FIG. 3B.

Coculture of Denuded Embryo with Purified ES Cells in an Eppendorf Micro Test Tube

Sexually mature albino ICR female mice were ip injected with 10 units of pregnant mare serum gonadotropin followed 48-64 hr later by 10 units of human chorionic gonadotropin, then paired with colored, stud B6CBAF1 males. The vaginal plug was checked the next morning. The superovulated 2.5 dpc donor embryos were flushed out from oviducts by modified KSOM-AA medium supplemented with 20.85 mM HEPES. Meanwhile, the zona pellucida of 8-cell embryos was removed with acidified Tyrode or pronase solution. The denuded embryos were washed and placed in modified KSOM-AA (supplemented with 1% FBS) droplets under light weight paraffin oil at 37° C., 5% CO₂ incubator until vial coculturing with ES cells.

A 0.8 mL purified ES cells suspension, ESC 26GJ9012-8-2, in 3.0˜5.0×10⁵/mL STO medium was added to an autoclaved, colorless 1.5 mL Eppendorf micro test tube. After standing for 5 min, 10-100 denuded 8-cell embryos were gently and circularly blown from beneath the medium surface into the vial via a mouth pipette. The coculturing Eppendorf micro test tube then were put back in the CO₂ incubator and stood for 2±1 hr. The probability of adhesion between embryos and ES cells, or between embryos themselves is increased if the concentration of ES cells is higher or if the incubation time is longer during the Eppendorf micro test tube coculture step. Additionally, when more denuded embryos are in a vial, the chance of two or more embryos stucking together increases as well.

Development of Chimeric Embryo After Cultured Overnight

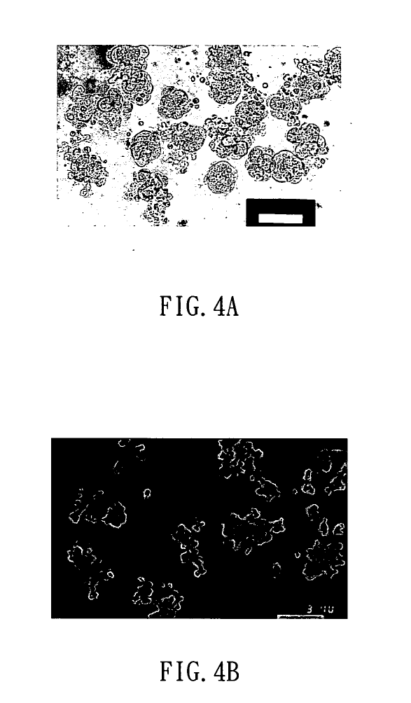

The precipitation in the vials was aspirated out gently and embryos adherent with ES cell were recovered and the loose cells on the surface of the “embryo-cell aggregate” were washed out with STO medium using mouth pipette. FIG. 4A (scale: 100 μm) shows 14 embryos wherein 4 of them being normal embryos with intact zona pellucida were used as controls. The remaining 10 embryos were denuded embryos, existing in the form of single, pairs or triplets. FIG. 4B (scale: 100 μm), which is of the same field as FIG. 4A, shows the green fluorescent ES cells attached to the surfaces of the embryo-cell aggregates. The embryo-cell aggregates with small amounts of STO medium then were directly transferred to modified KSOM-AA (supplemented with 1% FBS) droplets under light weight paraffin oil on bacteriological dishes and cultured overnight in 5% CO₂ incubator.

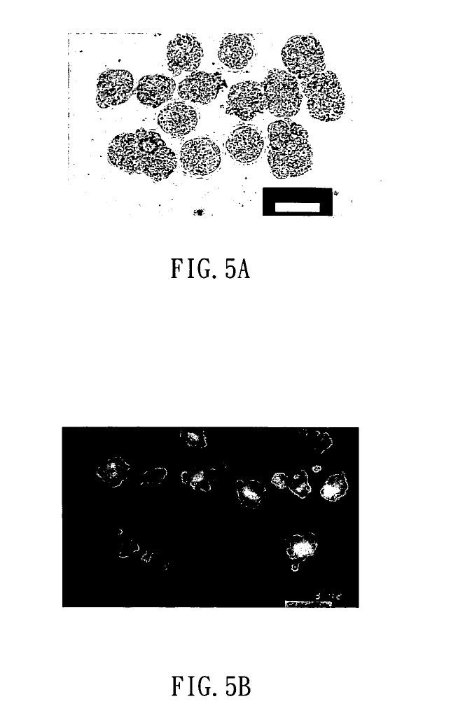

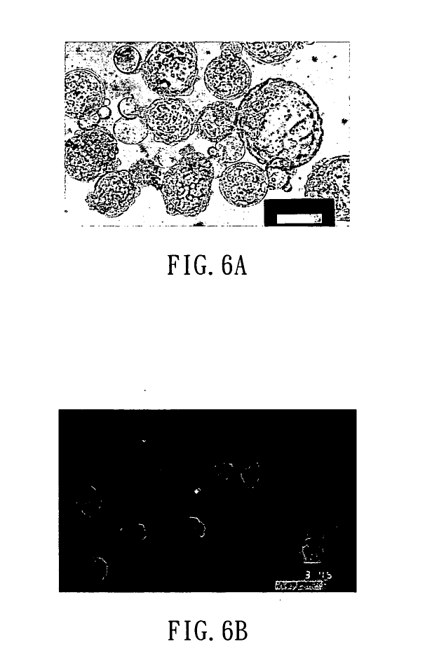

Following overnight in vitro culture, more than 80% of denuded embryo-ES cell aggregates developed to morula or early stage blastocysts which expressed green fluorescence and were comparable to control embryos with or without zona pellucida in this investigation as shown in FIG. 5A (scale: 100 μm).-The green fluorescent ES cells those originally attached on the surface of aggregates were incorparated inside the embryos to form chimeric morulas. Four normal embryos with zona pellucida as controls had grown into 3 early blastocysts and a morula. FIG. 5B (scale: 100 μm) shows the same view with FIG. 5A. Most of the ES cells on the surface of aggregates could internalize and reallocate into inner cell mass (ICM) after culturing to blastocysts, as shown in FIG. 6A and FIG. 6B (both show the same view with the scales being 100 μm), in which 4 normal embryo controls had grown into hatching blastocysts.

Chimeric Embryo Transfer

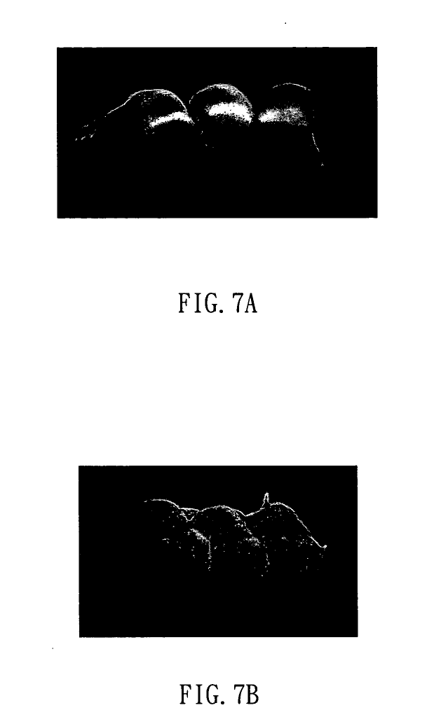

After culturing overnight, the successful and developing chimeric morula and/or blastocysts were transferred to pseudopregnant ICR using either 0.5 dpc oviduct or 2.5 dpc uterine horns. The female mouse gave birth after 19 or 17 days of pregnancy in average. FIG. 7A shows 3 chimeric mice naturally born after 12 chimeric blastocysts were transferred into the oviduct of a pseudopregnant ICR 0.5 dpc oviducts. Three farrowed pups with age of five days showing 100% coat color contribution and expressing green fluorescence (FIG. 7B).

Germline Transmission Capability of the Chimeric Mouse

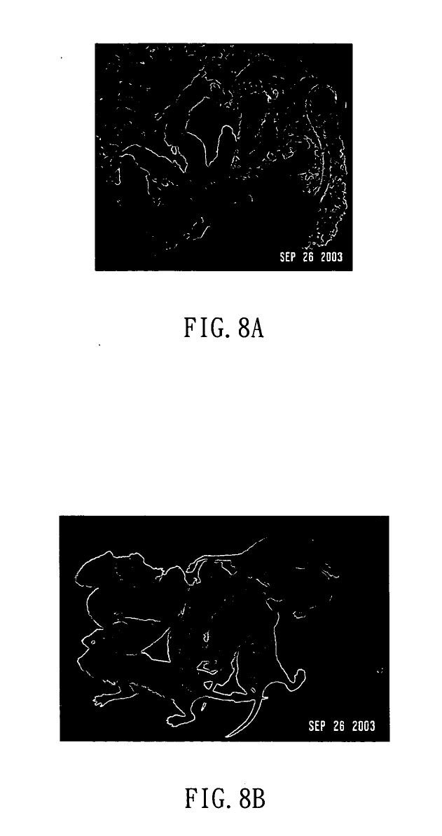

Phenotypically normal male chimeras with high contribution of coat color and expressing green fluorescent were naturally mated to female ICR. The coat color and/or green fluorescent expression of the farrowing pups then was checked for germline transmission. Fourteen pups were born, in which 9 expressed green fluorescent (shown in FIG. 8A and FIG. 8B), indicating the chimeric mouse was capable of germline transmission.

Coculture of Denuded 4n Embryo and Purified ES Cell in an Eppendorf Micro Test Tube

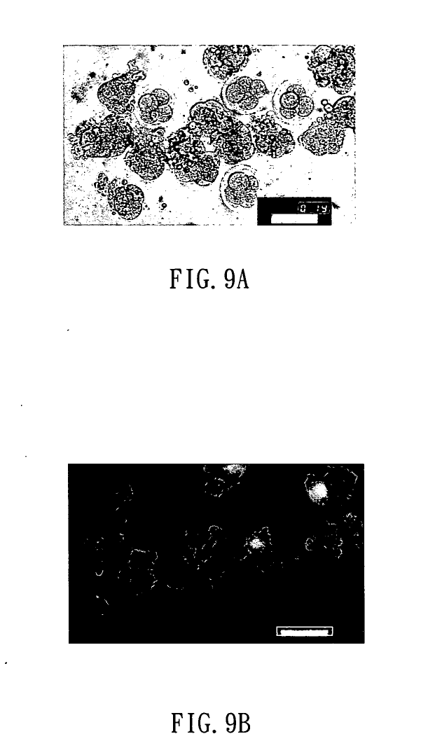

FIG. 9A (scale: 100 μm) shows that the embryo-cell aggregates recovered from the bottom of a 1.5 mL Eppendorf micro test tube after 2-hour coculture of electrofused 4n denuded embryos with purified mouse ESC 26GJ9012-8-2 (P15). Green fluorescent ES cells can be seen attaching to the surface of the aggregates. There were 19 embryos in total, in which 4 were 4n 4-cell embryos with intact zona pellucida (as control), 8 were single embryos, 2 were aggretages of 2 embryos, and 1 was aggregates of 3 embryos. FIG. 9B (scale: 100 μm) shows the same view with FIG. 9A under fluorescence microscopy.

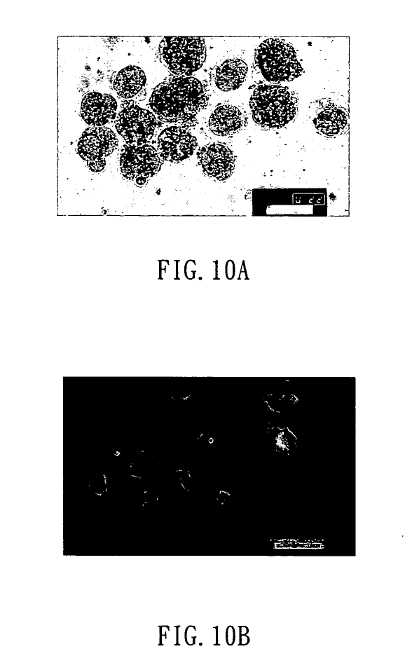

FIG. 10A (scale: 100 μm) shows that after overnight culture of the 4n embryo-cell aggregates shown in FIG. 9A, the green fluorescent ES cells originally attached to the surface of aggregates were incorparated into the embryos during the process of growning into morulas. The four control 4n embryos with zona pellucida had grown into four morulas. An embryo was grown from 3 adhered embryos, three embryos were grown from 2 attached embryos, and six embryos were grwon from single embryos. FIG. 10B (scale: 100 μm) is a fluorescent diagram showing the same field of that of FIG. 10A.

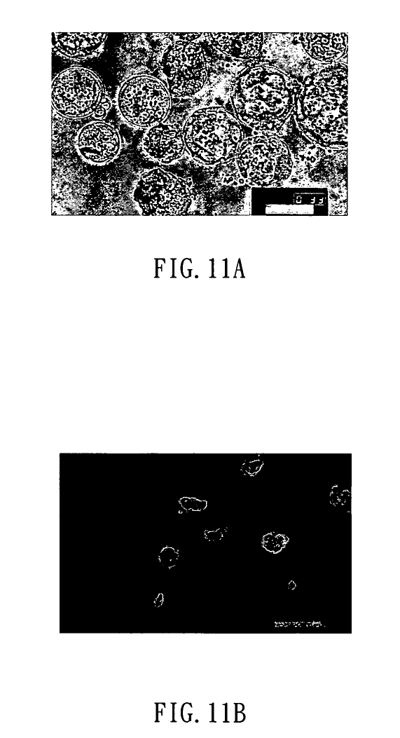

FIG. 11A (scale: 100 μm) shows the chimeric blastocysts were developed from the chimeric 4n morulas as shown in FIG. 10A after another overnight culturing. The green fluorescent ES cells mainly distributed in the inner cell mass. The four 4n morula controls had grown into hatching blastocysts. FIG. 11B (scale: 100 μm) is a fluorescent diagram showing the same field of that of FIG. 11A.

The present example shows that coculturing of denuded 4n embryos with purified mouse ES cells in an Eppendorf micro test tube generates chimeric embryo. When being transferred into the uterine horn of a ICR pseudopregnant female mouse, embryonic stem cell-derived mice are naturally born and express green fluorescence.

Observation and Photography

A Zeiss Axiovert 35 invert fluorescent microscopy system was used to observe and photograph the green fluorescent cells and embryos in the present examples. An OSRAM HBO 50 W/AC, 200 V high voltage mercury arc lamp was used as the light source. The best excitation range for EGFP is 488-490 nm; emission range is 507-509 nm (best range for green light is 520-530 nm). The fluorescent filter set 09, Cat. No. 487909 used in the example contains BP 450-490 exciter filter, FT 510 dichromatic beam splitter and LP 520 barrier filter. UVG-50 (Spectronics Co., Westbury N.Y., USA) UV goggles were used during fluorescent observation.

For the microscopy photographing, Kodak Ektachrome P1600 color reversal film with an ASA value of 400 or 800 set, or Kodak Ektachrome 400 film was used. The camera used was Contax 167/MT, allowing extended automatic exposure compensation (+2, shockproof system could be used), or a Zeiss MC100 system. The samples were placed in a culture plate or on a cover slip and covered with buffered saline.

For photographing the green fluorescent mice, the film used is as previously described. A long wavelength (365 nm) UV lamp purchased from UVP (http://www.uvp.com; Upland, Calif., USA) with blue glass shield was used as the light source (more than one lamps can be used to increase the light intensity). The camera body was a Nikon F-401 with a Nikon AF MICRO NIKKOR (55 mm, 1:2.8) lense, and a yellow filter was used for photograph.

In conclusion, the present invention provides a method for generating chimeric embryos, fetuses, and chimeric mammals, which avoids the problems of prior art while maintaining the advantages. The method involves coculture denuded, 2n 1-cell stage to morula stage or 4n 3-cell stage to morula stage embryos with ES cells in an Eppendorf micro test tube.

An Eppendorf micro test tube was chosen because it has the advantages of pointing bottom, wide opening with lid, suitable for autoclave and easy as well as cheap to purchase. The Eppendorf micro test tube coculture system adopted in this invention demonstrates that around 95% recovered denuded embryos adherent ES cells more or less tightly. The efficiency is clearly better than in previous coculture reports (63.9%: Wood et al., 1993b; 55%: Ueda et al., 1995), which denuded embryos lying on the dish surface basically only had a two-dimensional chance to contact ES cells. In the Eppendorf micro test tube coculture system of the present invention, the denuded embryos are surrounded three-dimensionally by ES cells during the 1˜3 hr coculture period. In fact, the cell adherent percentage could be 100% if the concentration of ES cells is increased or the coculture period is longer (data not shown).

Freshly thawed mouse embryonic stem cells can be used in the present invention so as to avoid routine, expensive, laborious, and time-consuming culture procedure. The method of the present invention is highly efficient (around 200 embryos can be processed at the same time by only one technician), low cost, and mass producible. It improves the technique of generating chimeric embryos. Moreover, the present invention is applicable to non-human mammals other than mice to produce chimeric embryos, and thus has wide application scope in the industry.

While the invention has been particularly shown and described with the reference to the preferred embodiment thereof, it will be understood by those skilled in the art that various changes in form and details may be made without departing from the spirit and scope of the invention.

Cited References

A Guidebook for the Care and Use of Laboratory Animals. 2001. The Chinese Association of Laboratory Animal Science, Taipei, Taiwan. (in Chinese)

Lee, K. H., 1992. Establishment of mouse embryonic stem cell line. Journal of the Chinese Society of Animal Science 21:267-282. (in Chinese with English tables, figures and abstract)

Lee, K. H., C. F. Tu, S. C. Wu, W. C. Hsu. 2003. Routine generation of green fluorescent chimeric mouse with a high percentage of germlinetransmission by a novel embryonic stem cell line. Journal of the Chinese Society of Animal Science, 32:143-154. (in Chinese with English tables, figures and abstract)

Biggers, J. D., L. K. McGinnis and M. Raffin. 2000. Amino acids and preimplantation development of the mouse in protein-free potassium simplex optimized medium. Biol. Reprod. 63:281-293.

Bradley, A. 1987. Production and analysis of chimeric mice. In: Robertson, E. J., ed. Teratocarcinomas and Embryonic Stem Cells, IRL Press, Oxford, pp. 113-152.

Erbach, G. T., J. A. Lawitts, V. E. Papaioannou and J. D. Biggers. 1994. Differential growth of the mouse preimplantation embryo in chemically defined media. Biol. Reprod. 50:1027-1033.

Hogan, B., R. Beddington, F. Costantini and E. Lacy. 1994. Manipulating the Mouse Embryo. Cold Spring Harbor Lab. Press, Cold Spring Harbor, Plainview, N.Y., USA.

Khillan, J. S. and Y. Bao. 1997. Preparation of animals with a high degree of chimerism by one-step coculture of embryonic stem cell and preimplantation embryos. BioTechniques 22:544-549.

Kondoh, G., Y. Yamamoto, K. Yoshida, Y. Suzuki, S. Osuka, Y. Nakano, T. Morita and J. Takeda. 1999. Easy assessment of ES cell clone potency for chimeric development and germ-line competency by an optimized aggregation method. J. Biochem. Biophys. Methods 39:137-142.

Nagy, A., M. Gertsenstein, K. Vintersten and R. Behringer. 2003. Manipulating the Mouse Embryo, a Laboratory Manual. 3rd ed. Cold Spring Harbor Lab. Press. Cold Spring Harbor, Plainview, N.Y., USA.

Niwa, H., K. Yamamura and J. Miyazaki. 1991. Efficient selection for high-expression transfectants with a novel eukaryotic vector. Gene 108:193-199.

Okabe, M., M. Ikawa, K. Kominami, T. Nakanishi and Y. Nishimune. 1997. ‘Green mice’ as a source of ubiquitous green cells. FEBS Lett. 407:313-319.

Papaioannou, V. and R. Johnson, 1993. Production of chimeras and genetically defined offspring from targeted ES cells. In Joyner, A. L. ed. Gene Targeting: A Practical Approach. IRL Press, Oxford Univ. Press, Oxford, pp. 107-146.

Papaioannou, V. and R. Johnson, 2000. Production of chimeras by blastocyst and morula injection of targeted ES cells. In Joyner, A. L. 2nd ed. Gene Targeting: A Practical Approach. Oxford Univ. Press, Oxford, pp. 132-175.

Shimada, H., T. Kaname, M. Suzuki, Y. Hitoshi, K. Araki, T. Imaizumi and K. I. Yamamura. 1999. Comparison of ES cell fate in sandwiched aggregates and co-cultures aggregates during blastocyst formation by monitored GFP expression. Mol. Reprod. Dev. 52:376-382.

Suzuki, H., N. Kamada, O. Ueda, K. Jishage, H. Kurihara, Y. Kurihara, T. Kodama, Y. Yazaki, S. Azuma and Y. Toyoda. 1994. Production of a germ-line chimera by coculture of zona-free embryos with frozen-thawed embryonic stem cells. J. Reprod. Dev. 40:361-365.

Tokunaga, T. and Y. Tsunoda. 1992. Efficacious production of viable germ-line chimeras between embryonic stem (ES) cells and 8-cell stage embryos. Develop. Growth Differ. 34:561-566.

Tsien, R. Y. 1998. The green fluorescent protein. Annu. Rev. Biochem.67:509-544.

Ueda, O, N. Kamada, K. Jishage, S. Uchida and H. Suzuki. 1995. Factors affecting the efficiency of chimera production by coculture of zona-free embryos with frozen-thawed embryonic stem cells in mice. J. Reprod. Dev. 41:181-186.

Wood, S. A., N. D. Allen, J. Rossant, A. Auerbach and A. Nagy. 1993a. Non-injection methods for the production of embryonic stem cell-embryo chimaeras. Nature 365:87-89.

Wood, S. A., W. S. Pascoe, C. Schmidt, R. Kemler, M. J. Evans and N. D. Allen.1993b.Simple and efficient production of embryonic stem cell-embryo chimeras by coculture. Proc. Natl. Acad. Sci. USA 90:4582-4585.

1. A method for generating a non-human mammalian chimeric embryo, comprising the following steps:
providing a cell;
providing a denuded non-human mammalian embryo that is from 1-cell stage to morula stage;
mixing and coculturing said cell and said denuded non-human mammalian embryo in physiological medium in an Eppendorf micro test tube to obtain a non-human mammalian embryo-cell aggregate; and
continuing culturing said non-human mammalian embryo-cell aggregate in physiological medium so as to generate a non-human mammalian chimeric embryo.

2. The method according to claim 1, further comprising the following steps:
transferring said non-human mammalian chimeric embryo into a non-human recipient mammal; and
allowing said transferred chimeric embryo to grow in said non-human recipient mammal so as to develop into an offspring.

3. The method according to claim 2, wherein said offspring comprises a non-human chimeric fetus or an embryonic stem cell-derived fetus.

4. The method according to claim 2, wherein said offspring comprises a non-human chimeric mammal or an embryonic stem cell-derived mammal.

5. The method according to claim 1, wherein said physiological medium used for step of coculturing said cell and said denuded non-human mammalian embryo is further supplemented with serum or binder.

6. The method according to claim 5, wherein said binder is lectins.

7. The method according to claim 1, wherein said physiological medium used for the step of culturing said non-human embryo-cell aggregate or said non-human mammalian chimeric embryo is further supplemented with serum.

8. The method according to claim 1, wherein said cell is obtained from a cell line or a primary cell.

9. The method according to claim 1, wherein said cell is treated or untreated with purification procedures.

10. The method according to claim 1, wherein said cell is a cell whose genetic material has or has not been modified.

11. The method according to claim 1, wherein said denuded non-human mammalian embryo comprises diploid or multiploid chromosome.

12. The method according to claim 1, wherein said denuded non-human mammalian embryo is obtained by way of in vivo growth, in vitro culture system, or a combination thereof.

13. The method according to claim 1, wherein said cell and said denuded non-human mammalian embryo are of the same species or different species.

14. The method according to claim 1, wherein said Eppendorf micro test tube is a sterilizable, hollow container with or without lid and without any particular specifications.

15. The method according to claim 2, wherein said chimeric embryo is transferred into an oviduct, a uterus, or a uterine horn of a non-human recipient mammal.

16. A method for generating non-human chimeric mammal, comprising the following steps:
providing a cell;
providing a denuded non-human mammalian embryo that is from 1-cell stage to morula stage;
mixing and coculturing said cell and said denuded non-human mammalian embryo in physiological medium in an Eppendorf micro test tube to obtain a non-human mammalian embryo-cell aggregate;
continuing culturing said non-human mammalian embryo-cell aggregate in physiological medium so as to generate a non-human mammalian chimeric embryo;
transferring said non-human mammalian chimeric embryo into a non-human recipient mammal; and
allowing said transferred chimeric embryo to grow in said non-human recipient mammal so as to develop to term as a non-human chimeric mammal or an embryonic stem cell-derived mammal.

17. A method for generating non-human chimeric fetus, comprising the following steps:
providing a cell;
providing a denuded non-human mammalian embryo that is from 1-cell stage to morula stage;
mixing and coculturing said cell and said denuded non-human mammalian embryo in physiological medium in an Eppendorf micro test tube to obtain a non-human mammalian embryo-cell aggregate;
continuing culturing said non-human mammalian embryo-cell aggregate in physiological medium so as to generate a non-human mammalian chimeric embryo;
transferring said non-human mammalian chimeric embryo into a non-human recipient mammal; and
allowing said transferred chimeric embryo to grow in said non-human recipient mammal so as to develop into a non-human chimeric fetus or an embryonic stem cell-derived fetus.
